(19) United States
(12) Patent Application Publication
(10) Pub. No.: US 2017/0290572 A1
(43) Pub. Date: Oct. 12, 2017

(54) METHOD FOR COLLECTING TISSUE

(71) Applicant: OLYMPUS CORPORATION, Tokyo (JP)

(72) Inventors: Mari NAKATA, Tokyo (JP); Hiromi SANUKI, Yokohama-shi (JP); Junichi MURAMATSU, Sagamihara-shi (JP); Keita SHOJI, Tokyo (JP)

(73) Assignee: OLYMPUS CORPORATION, Tokyo (JP)

(21) Appl. No.: 15/094,789

(22) Filed: Apr. 8, 2016

(51) Int. Cl. A61B 10/02; A61B 8/12; A61M 5/32; A61B 10/00

(52) U.S. Cl. CPC A61B 10/0233; A61B 10/0045; A61B 8/12; A61M 5/32; A61M 5/3202; A61B 2010/0061; A61B 2010/0216

(57) ABSTRACT

A method for collecting tissue includes a body fluid removal step in which a body fluid inside a pancreatic cyst is moved to an outside of the pancreatic cyst, an injecting step in which a liquid different from the body fluid is injected into the pancreatic cyst, a collecting step in which the liquid is collected from the pancreatic cyst, and a separating step in which tissue of an inner wall of the pancreatic cyst, which is contained in the liquid collected from the pancreatic cyst, is separated from the liquid.

### BACKGROUND OF THE INVENTION

### Field of the Invention

The present invention relates to a method for collecting tissue. More specifically, the present invention relates to a method for collecting tissue in which tissue of an inner wall of a pancreatic cyst, which is called a cyst wall, is collected.

### Description of Related Art

As a method of performing discrimination of whether a cyst that develops in the pancreas (hereinafter referred to as a “pancreatic cyst”) is benign or malignant, a method of performing cytodiagnosis on a pancreatic cyst is known. In the related art, a body fluid inside the pancreatic cyst, which is called a cyst fluid, is collected, cells are separated from the collected body fluid, and therefore cells necessary for performing the cytodiagnosis are obtained. In addition, in a study by Al-Haddad et al. (Al-Haddad, M. et al. (2007), Safety and efficacy of cytology brushings versus standard FNA in evaluating cystic lesions of the pancreas: a pilot study. Gastrointestinal Endoscopy, 65(6), 894-898.), in addition to collecting cells from the body fluid of the pancreatic cyst, after the body fluid is aspirated from inside the pancreatic cyst, a brush is projected from a distal end of a puncture needle that has punctured the pancreatic cyst, an inner wall of the pancreatic cyst is rubbed with the brush, and therefore cells of the inner wall that adhere to the brush are collected.

### SUMMARY OF THE INVENTION

According to a first aspect of the present invention, a method for collecting tissue includes: a body fluid removal step in which a body fluid inside a pancreatic cyst is moved to an outside of the pancreatic cyst; an injecting step in which a liquid different from the body fluid is injected into the pancreatic cyst; a collecting step in which the liquid is collected from the pancreatic cyst; and a separating step in which tissue of an inner wall of the pancreatic cyst, which is contained in the liquid collected from the pancreatic cyst, is separated from the liquid.

According to a second aspect of the present invention, the method for collecting tissue according to the first aspect may further include an exfoliating step in which the inner wall of the pancreatic cyst is scraped and the tissue is exfoliated from the inner wall. The exfoliating step may be performed after the body fluid removal step, and the injecting step may be performed after the exfoliating step.

According to a third aspect of the present invention, in the method for collecting tissue according to the second aspect, in the body fluid removal step, the body fluid may be aspirated from the pancreatic cyst, and a lumen of the pancreatic cyst may decrease in size. In the exfoliating step, a treatment instrument capable of scraping the inner wall of the pancreatic cyst may be inserted into the decreased lumen, and the inner wall may be scraped by the treatment instrument.

According to a fourth aspect of the present invention, in the method for collecting tissue according to the first aspect, the liquid may have a property of exfoliating the tissue from the inner wall of the pancreatic cyst.

According to a fifth aspect of the present invention, in the method for collecting tissue according to the fourth aspect, the liquid may be one of normal saline, a protease solution, and a surfactant solution.

According to a sixth aspect of the present invention, the method for collecting tissue according to the first aspect may further include a perfusing step in which an inside of the pancreatic cyst is perfused with the liquid. The perfusing step may be performed after the injecting step, and the collecting step may be performed after the perfusing step.

### BRIEF DESCRIPTION OF THE DRAWINGS

FIG. 1 is a general view of an ultrasound endoscope and a puncture needle used in a method for collecting tissue according to a first embodiment of the present invention.

FIG. 2 is a cross-sectional view of a distal end portion of the ultrasound endoscope.

FIG. 3 is a general view of the puncture needle.

FIG. 4 is an entire cross-sectional view of the puncture needle.

FIG. 5 is a diagram showing a state in which a syringe is attached to the puncture needle.

FIG. 6 is a flowchart showing the method for collecting tissue.

FIG. 7 is a diagram showing an example of a pancreatic cyst.

FIG. 8 is a diagram showing the method for collecting tissue.

FIG. 9 is a diagram showing the method for collecting tissue.

FIG. 10 is a diagram showing the method for collecting tissue.

FIG. 11 is a diagram showing the method for collecting tissue.

FIG. 12 is a flowchart showing a method for collecting tissue according to a second embodiment of the present invention.

FIG. 13 is a diagram showing the method for collecting tissue.

FIG. 14A to FIG. 14C are diagrams showing an example of a treatment instrument used in the method for collecting tissue.

FIG. 15 is a diagram showing another example of the treatment instrument used in the method for collecting tissue.

FIG. 16 is a diagram showing still another example of the treatment instrument used in the method for collecting tissue.

FIG. 17A and FIG. 17B are diagrams showing yet another example of the treatment instrument used in the method for collecting tissue.

FIG. 18 is a diagram showing yet another example of the treatment instrument used in the method for collecting tissue.

FIG. 19 is a diagram showing yet another example of the treatment instrument used in the method for collecting tissue.

### DETAILED DESCRIPTION OF THE INVENTION

### First Embodiment

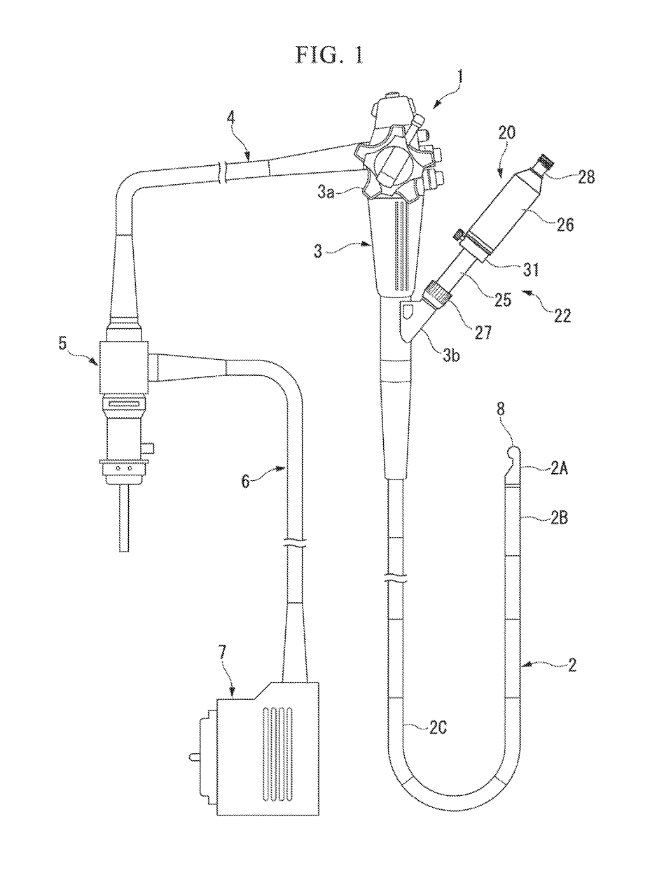

Hereinafter, a first embodiment of the present invention will be described with reference to FIG. 1 to FIG. 11.

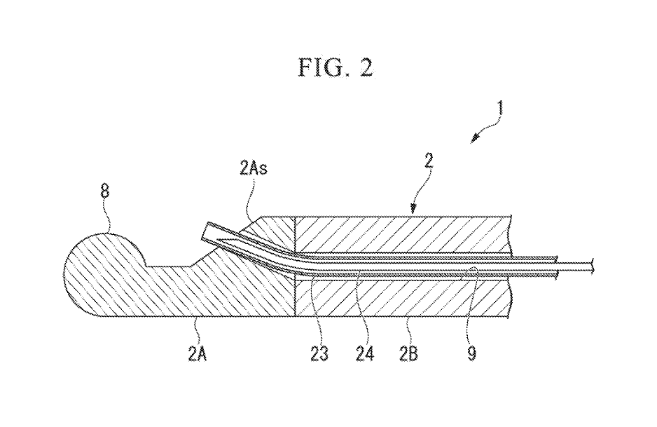

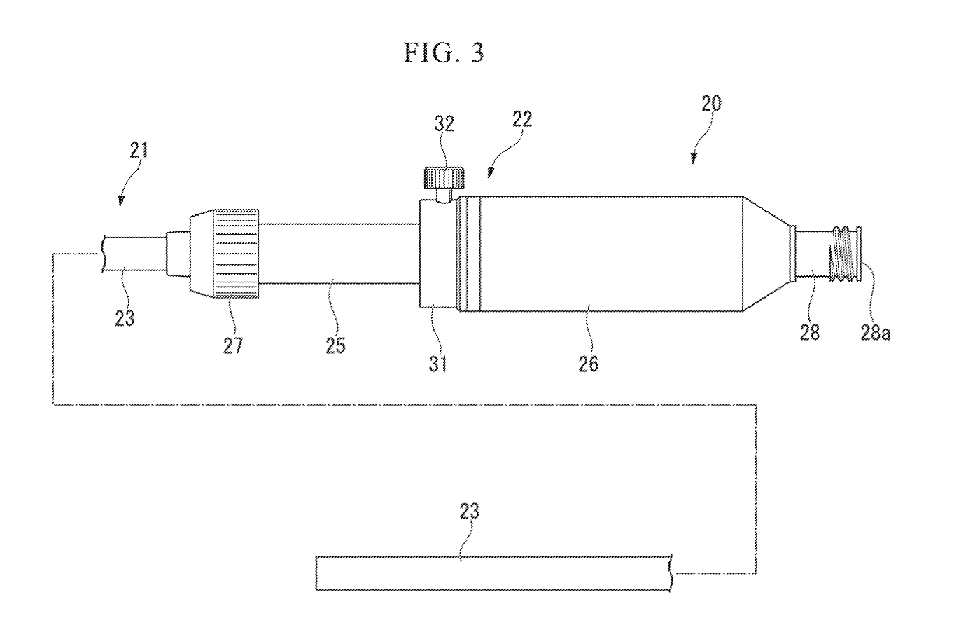

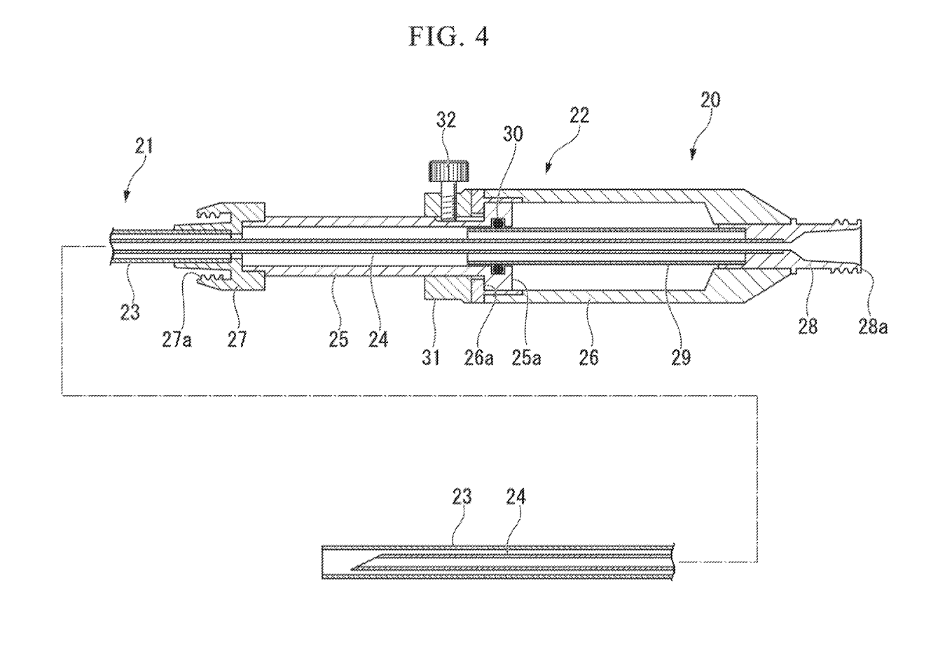

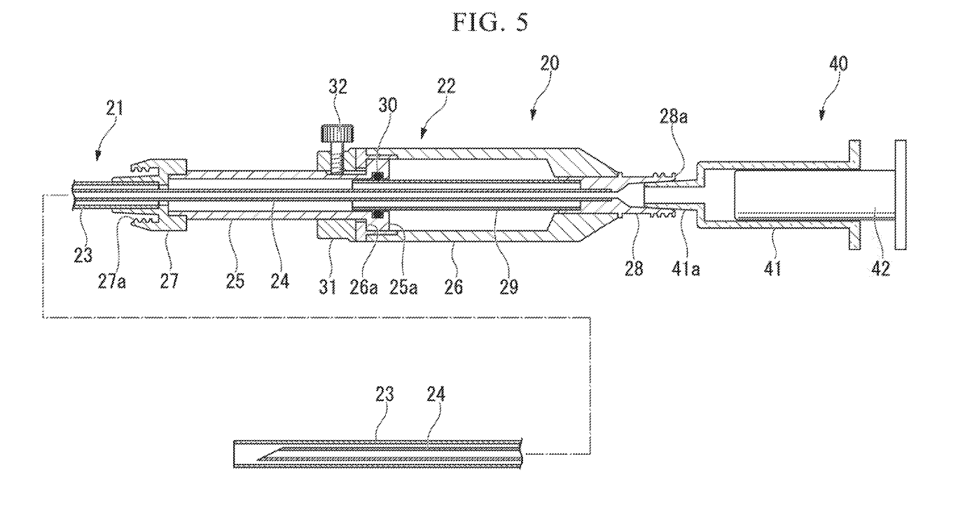

First, an ultrasound endoscope 1 and a puncture needle 20 used in a method for collecting tissue according to the present embodiment will be described. FIG. 1 is a general view of the ultrasound endoscope 1 and the puncture needle 20. FIG. 2 is a cross-sectional view of a distal end portion of the ultrasound endoscope 1. FIG. 3 is a general view of the puncture needle 20. FIG. 4 is an entire cross-sectional view of the puncture needle 20. FIG. 5 is a diagram showing a state in which a syringe 40 is attached to the puncture needle 20.

The ultrasound endoscope 1 includes an elongated insertion portion 2 that is inserted into a body cavity, an operation unit 3 provided at a proximal end of the insertion portion 2, and a universal cord 4 having one end that is connected to a side portion of the operation unit 3.

An endoscope connector 5 is provided at the other end of the universal cord 4. One end of an ultrasound cable 6 is connected to a side portion of the endoscope connector 5. An ultrasound connector 7 is provided at the other end of the ultrasound cable 6.

The insertion portion 2 includes a distal end rigid portion 2A, a bending portion 2B and a flexible tube portion 2C. The distal end rigid portion 2A is formed of a rigid member. The distal end rigid portion 2A includes an ultrasound scanning mechanism 8 for ultrasound observation and an optical imaging mechanism (not shown) for optical observation.

The ultrasound scanning mechanism 8 includes an ultrasound vibrator (not shown) configured to emit and receive ultrasound waves. In the ultrasound scanning mechanism 8, ultrasound waves that are emitted from the ultrasound vibrator and collide with and are reflected by an observation target are received by the ultrasound vibrator as reflection waves. In addition, a signal cable (not shown) is connected to the ultrasound scanning mechanism 8. The signal cable extends to the ultrasound connector 7 through the insertion portion 2, the operation unit 3, the universal cord 4, the endoscope connector 5, and the ultrasound cable 6.

The ultrasound connector 7 is connected to an ultrasound observation device (not shown). The ultrasound scanning mechanism 8 outputs a signal based on the ultrasound wave received by the ultrasound vibrator to the ultrasound observation device through the signal cable. The ultrasound observation device converts the received signal into an ultrasound image and displays the image on a monitor (not shown).

A signal cable (not shown) is connected to the optical imaging mechanism. The signal cable extends to the endoscope connector 5 through the insertion portion 2, the operation unit 3, and the universal cord 4. An endoscopic observation device (not shown) is connected to the endoscope connector 5. The endoscopic observation device displays the signal output from the optical imaging mechanism on a monitor (not shown) as an observation image.

The bending portion 2B is connected to a proximal end side of the distal end rigid portion 2A and is configured to be bendable. An operation wire (not shown) is connected to the bending portion 2B. The operation wire extends to the operation unit 3 from the bending portion 2B through the insertion portion 2, and is connected to an angle knob 3a provided in the operation unit 3. When the angle knob 3a is operated to pull the operation wire, it is possible to bend the bending portion 2B.

The flexible tube portion 2B is an elongated member that extends from a proximal end of the bending portion 2B to a distal end of the operation unit 3 and has flexibility.

A channel 9 into which a treatment instrument such as the puncture needle 20 can be inserted is provided inside the insertion portion 2. A distal end of the channel 9 is opened to a distal end surface 2As of the distal end rigid portion 2A. A proximal end of the channel 9 communicates with an insertion opening 3b provided in the operation unit 3. A proximal end portion of the insertion opening 3b is formed in a Luer lock shape that can connect the treatment instrument. The treatment instrument is inserted into the channel 9 from the insertion opening 3b, and passes through the channel 9. Therefore, a distal end of the treatment instrument can be projected from the distal end surface 2As of the distal end rigid portion 2A.

As shown in FIG. 3 and FIG. 4, the puncture needle 20 includes an insertion portion 21 and an operation unit 22. The insertion portion 21 is a portion that is inserted into the channel 9 of the ultrasound endoscope 1. The operation unit 22 is provided at a proximal end portion of the insertion portion 21 and can be fixed to the insertion opening 3b of the ultrasound endoscope 1.

The insertion portion 21 includes a sheath 23 and a needle tube 24. The sheath 23 is a tube having flexibility. The needle tube 24 is inserted into the sheath 23 such that the needle tube 24 can be advanced and retracted in the sheath 23. The needle tube 24 is formed of, for example, an elongated and thin stainless steel pipe. A distal end of the needle tube 24 is formed in a sharp shape.

The operation unit 22 includes an operation unit main body 25 and a slider 26. The operation unit main body 25 is formed in an elongated cylindrical shape. A connection portion 27 is provided at a distal end portion of the operation unit main body 25. The sheath 23 is fixed to the connection portion 27. In addition, a threaded portion 27a for connection to the insertion opening 3b of the ultrasound endoscope 1 is formed in the connection portion 27. When the threaded portion 27a is screwed to the proximal end portion of the insertion opening 3b, it is possible to fix the puncture needle 20 to the ultrasound endoscope 1. In addition, a flange portion 25a to be engaged with the slider 26 is formed at a proximal end portion of the operation unit main body 25.

The slider 26 is provided to be slidable in a longitudinal direction with respect to the operation unit main body 25. The slider 26 is formed in a cylindrical shape and has an inside into which the operation unit main body 25 can be inserted. A locking portion 26a to be locked to the flange portion 25a is provided at a distal end portion of the slider 26. A connection base member 28 is provided at a proximal end portion of the slider 26. A proximal end portion of the needle tube 24 and a proximal end portion of a guide pipe 29 disposed inside the slider 26 are fixed to a distal end portion of the connection base member 28. A proximal end portion of the connection base member 28 is formed in a Luer lock shape that can connect, for example, the treatment instrument to be inserted into the needle tube 24. An opening 28a of the proximal end portion of the connection base member 28 communicates with a lumen of the needle tube 24.

The guide pipe 29 is formed in a cylindrical shape. A distal end portion of the guide pipe 29 is held in an O-ring 30 provided in an inner circumferential side of the flange portion 25a.

A stopper 31 is provided at the operation unit main body 25. The stopper 31 is formed in a cylindrical shape, and is slidably disposed in the longitudinal direction with respect to the operation unit main body 25. A fixation screw 32 to be screwed into the stopper 31 is provided at the stopper 31. By abutting the fixation screw 32 on an outer circumferential surface of the operation unit main body 25, the stopper 31 can be fixed to the operation unit main body 25. In addition, when the stopper 31 is fixed to a desired position in the longitudinal direction of the operation unit main body 25, it is possible to set the sliding distance of the slider 26.

As shown in FIG. 5, the syringe 40 can be attached to the proximal end portion of the connection base member 28 of the puncture needle 20. The syringe 40 includes a cylinder 41 having a cylindrical shape and a piston 42 that is slidably inserted into the cylinder 41. A cylinder connection base 41a that can be attached to the proximal end portion of the connection base member 28 is formed at a distal end portion of the cylinder 41.

By pushing the piston 42 while the cylinder 41 is filled with a liquid, it is possible to release the liquid into the body through the lumen of the needle tube 24. In addition, by pulling the piston 42 while the cylinder 41 is empty, it is possible to aspirate a body fluid from inside the body through the distal end of the needle tube 24.

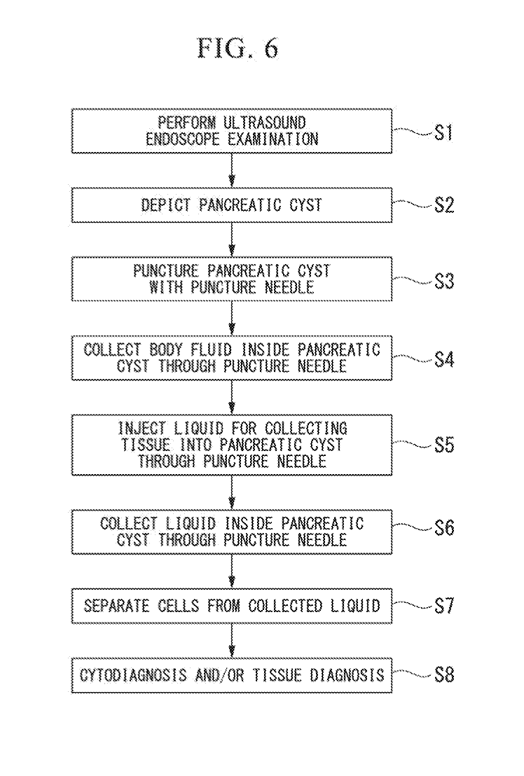

Next, a method for collecting tissue according to the present embodiment will be described with reference to FIG. 6 to FIG. 11.

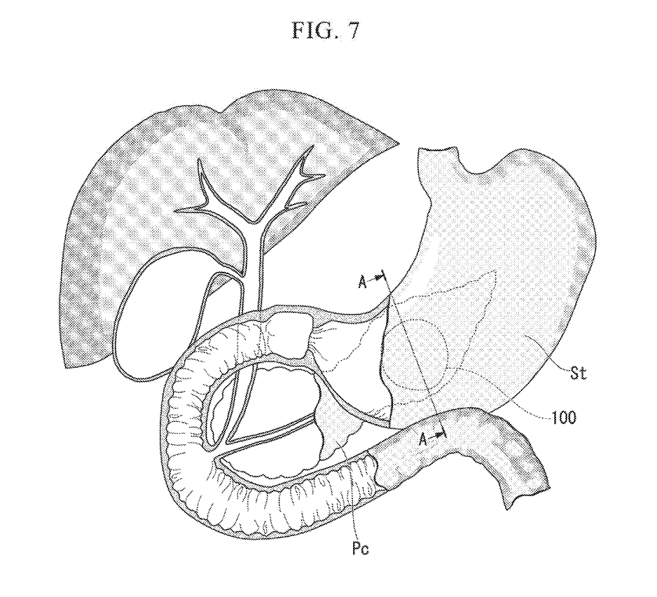

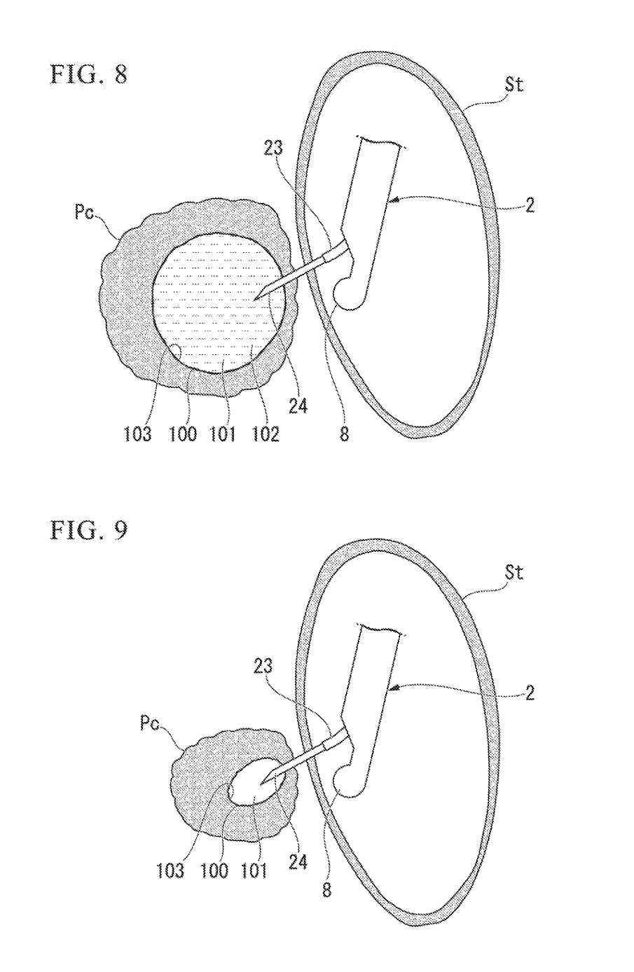

FIG. 6 is a flowchart showing the method for collecting tissue. FIG. 7 is a diagram showing an example of a pancreatic cyst. FIG. 8 to FIG. 11 are diagrams showing the method for collecting tissue, and show a cross section taken along the line A-A in FIG. 7. Hereinafter, as shown in FIG. 7, an example in which a pancreatic cyst 100 is formed in the tail of the pancreas Pc will be described.

First, an endoscopist inserts the sheath 23 of the puncture needle 20 from the insertion opening 3b of the ultrasound endoscope 1, and screws the threaded portion 27a formed in the connection portion 27 of the puncture needle 20 to the proximal end portion of the insertion opening 3b. Accordingly, the puncture needle 20 is fixed to the ultrasound endoscope 1.

Next, the insertion portion 2 of the ultrasound endoscope 1 is inserted into the stomach St. The inside of the stomach St is subjected to ultrasonography (Step S1). The pancreatic cyst 100 is depicted in an ultrasound image (Step S2). While a position of the pancreatic cyst 100 is confirmed in the ultrasound image, the endoscopist slides the slider 26 of the puncture needle 20, and causes the needle tube 24 to penetrate through a stomach wall to puncture the pancreatic cyst 100 (Step S3). Therefore, as shown in FIG. 8, the distal end of the needle tube 24 is disposed in a lumen 101 of the pancreatic cyst 100.

The empty syringe 40 is attached to the proximal end portion of the connection base member 28 of the puncture needle 20. The piston 42 of the syringe 40 is pulled with respect to the cylinder 41, and a body fluid 102 is aspirated from inside the pancreatic cyst 100. Accordingly, the body fluid 102 is collected inside the cylinder 41 from the distal end of the needle tube 24 through the lumen of the needle tube 24 (Step S4, body fluid removal step). In addition, when the body fluid 102 is aspirated from the inside of the pancreatic cyst 100, the lumen 101 of the pancreatic cyst 100 decreases in size, as shown in FIG. 9.

The syringe 40 in which the body fluid 102 is collected is removed from the connection base member 28 of the puncture needle 20. The syringe 40 filled with a liquid 43 for collecting tissue, which is a liquid different from the body fluid 102, is attached to the proximal end portion of the connection base member 28 of the puncture needle 20. The liquid 43 is harmless to the human body, and has a property of exfoliating tissue of an inner wall 103 of the pancreatic cyst 100 from the inner wall 103 of the pancreatic cyst 100. Examples of the liquid 43 include normal saline, a protease solution, and a surfactant solution.

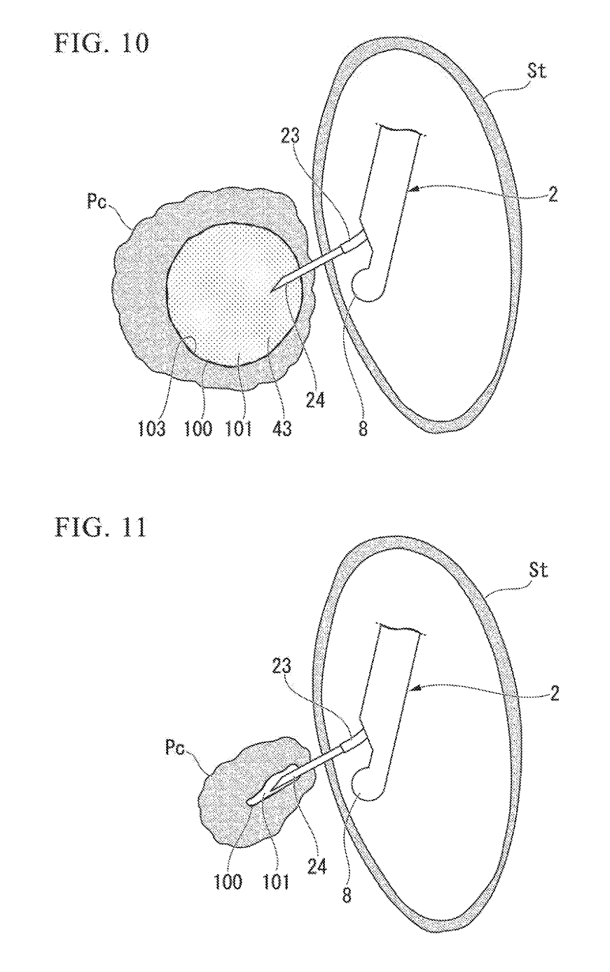

The piston 42 of the syringe 40 filled with the liquid 43 is pushed toward the cylinder 41, and the liquid 43 is injected into the pancreatic cyst 100 from the distal end of the needle tube 24, as shown in FIG. 10 (Step S5, injecting step). Since the liquid 43 has the above-described property, tissue of the inner wall 103 of the pancreatic cyst 100 is exfoliated from the inner wall 103 of the pancreatic cyst 100 by the liquid 43. Accordingly, the tissue of the inner wall 103 is contained in the liquid 43.

The piston 42 of the syringe 40 is pulled with respect to the cylinder 41 and the liquid 43 is aspirated from inside the pancreatic cyst 100. Accordingly, the liquid 43 and the tissue in the liquid 43 are collected inside the cylinder 41 from the distal end of the needle tube 24 through the lumen of the needle tube 24 (Step S6, collecting step). In addition, when the liquid 43 is aspirated from the inside of the pancreatic cyst 100, the lumen 101 of the pancreatic cyst 100 decreases in size, as shown in FIG. 11.

The tissue of the inner wall 103 of the pancreatic cyst 100 that is contained in the liquid 43 is separated from the liquid 43 collected from the pancreatic cyst 100 by a method such as centrifugation (Step S7, separating step). Accordingly, the tissue of the pancreatic cyst 100 is obtained. Pathological diagnosis, for example, predetermined cytodiagnosis or tissue diagnosis including immunohistochemistry, is performed on the obtained tissue (Step S8), and discrimination of whether the pancreatic cyst 100 is benign or malignant is performed. In addition, the obtained tissue can be used for examination of mutant genes such as a GNAS gene or a KRAS gene in addition to the pathological diagnosis.

While the body fluid 102 is aspirated from inside the pancreatic cyst 100 in the above Step 4, the present invention is not limited thereto. The body fluid 102 may be moved to the outside of the pancreatic cyst 100.

In addition, while the liquid 43 injected into the pancreatic cyst 100 is collected in the above Step 6, an inside of the pancreatic cyst 100 may be perfused with the liquid 43 before the liquid 43 is collected (perfusing step). For example, by pushing and pulling the piston 42 of the syringe 40 toward and away from the cylinder 41, it is possible to generate a flow of the liquid 43 inside the pancreatic cyst 100. As another method, a double-lumen catheter having two holes is inserted into the puncture needle 20, the liquid 43 is delivered through one hole and the liquid 43 is aspirated through the other hole. Therefore, it is possible to perform perfusion with the liquid 43.

The method for collecting tissue according to the present embodiment includes the body fluid removal step in which the body fluid 102 inside the pancreatic cyst 100 is moved to the outside of the pancreatic cyst 100, the injecting step in which the liquid 43 different from the body fluid 102 is injected into the pancreatic cyst 100, the collecting step in which the liquid 43 is collected from the pancreatic cyst 100, and the separating step in which tissue of the inner wall 103 of the pancreatic cyst 100 that is contained in the liquid 43 collected from the pancreatic cyst 100 is separated from the liquid 43.

According to the above-described method, the liquid 43 different from the body fluid 102 is injected into the pancreatic cyst 100, the liquid 43 is collected from the pancreatic cyst 100, and the tissue of the pancreatic cyst 100 is collected. Therefore, it is possible to stably obtain a lot of tissue and improve sensitivity of pathological diagnosis and genetic testing.

In addition, the liquid 43 has a property of exfoliating tissue from the inner wall 103 of the pancreatic cyst 100. Specifically, the liquid 43 is normal saline, a protease solution, or a surfactant solution. Accordingly, since the tissue of the pancreatic cyst 100 is exfoliated from the inner wall 103 of the pancreatic cyst 100 by the liquid 43, it is possible to collect the tissue of the pancreatic cyst 100 more efficiently.

In addition, the method for collecting tissue according to the present embodiment may further include the perfusing step in which the inside of the pancreatic cyst 100 is perfused with the liquid 43. The perfusing step is performed after the injecting step, and the collecting step is performed after the perfusing step. Therefore, since the inside of the pancreatic cyst 100 is perfused with the liquid 43, it is possible to collect the tissue of the pancreatic cyst 100 more efficiently.

### Second Embodiment

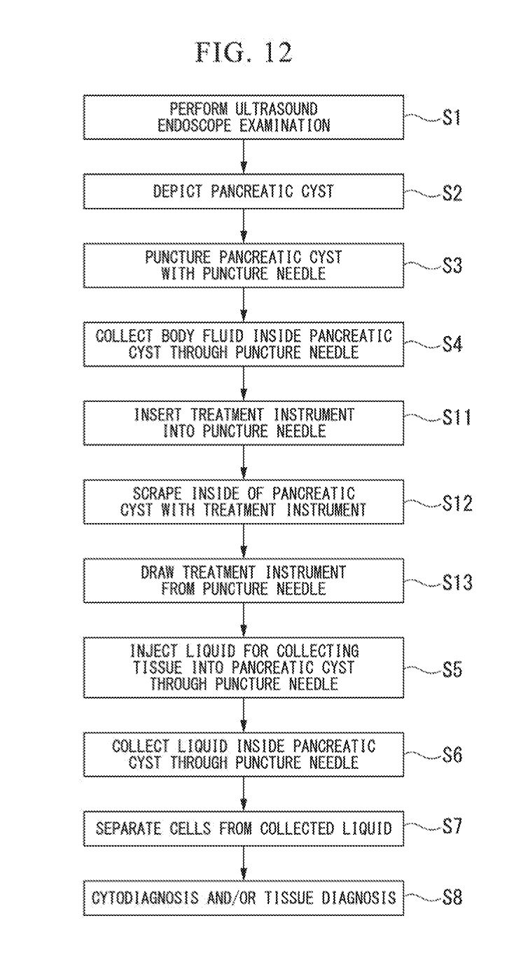

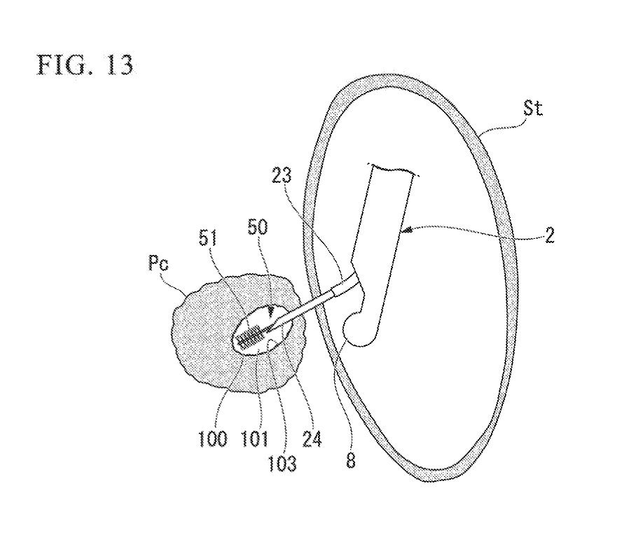

Next, a second embodiment of the present invention will be described with reference to FIG. 12 and FIG. 13.

FIG. 12 is a flowchart showing a method for collecting tissue according to the present embodiment. FIG. 13 is a diagram showing the method for collecting tissue.

The method for collecting tissue according to the present embodiment is different from the method for collecting tissue according to the first embodiment in that a step in which the inner wall 103 of the pancreatic cyst 100 is scraped and tissue is exfoliated from the inner wall 103 is further included. Details of parts having the same configuration as the method for collecting tissue according to the first embodiment will not be described.

In the method for collecting tissue according to the present embodiment, Step S1 to Step S4 are the same as those in the above-described method for collecting tissue according to the first embodiment. In Step 4, after the syringe 40 in which the body fluid 102 is collected is removed from the connection base member 28 of the puncture needle 20, a treatment instrument 50 having a distal end to which a brush-shaped treatment unit 51 is provided is inserted into the lumen of the needle tube 24 from the opening 28a of the connection base member 28 of the puncture needle 20, and is projected from the distal end of the needle tube 24, as shown in FIG. 13 (Step S1)). Accordingly, the treatment unit 51 of the treatment instrument 50 is disposed inside the lumen 101 of the pancreatic cyst 100.

By appropriately moving the treatment unit 51 of the treatment instrument 50 forward and backward and rotating it, the inner wall 103 of the pancreatic cyst 100 is scraped, and tissue of the inner wall 103 is exfoliated from the inner wall 103 (Step S12, exfoliating step). In this case, since the lumen 101 of the pancreatic cyst 100 decreases in size in Step S4, the treatment unit 51 comes in contact with the inner wall 103 with ease.

The treatment instrument 50 is drawn from the needle tube 24 of the puncture needle 20 (Step S13). Next, the syringe 40 filled with the liquid 43 for collecting tissue is attached to the proximal end portion of the connection base member 28 of the puncture needle 20, and the above Step S5 to Step S8 are performed.

The method for collecting tissue according to the present embodiment further includes the exfoliating step in which the inner wall 103 of the pancreatic cyst 100 is scraped and tissue is exfoliated from the inner wall 103. The exfoliating step is performed after the body fluid removal step, and the injecting step is performed after the exfoliating step.

Therefore, since the inner wall 103 of the pancreatic cyst 100 is scraped and tissue of the inner wall 103 is exfoliated from the inner wall 103, it is possible to collect more tissue compared to when only the liquid 43 is used to collect tissue. Further, since the liquid 43 is injected into the pancreatic cyst 100 after the tissue of the pancreatic cyst 100 is exfoliated, it is possible collect tissue more efficiently with the liquid 43.

In addition, in the body fluid removal step, the body fluid 102 is aspirated from the pancreatic cyst 100, and the lumen 101 of the pancreatic cyst 100 decreases in size. In the exfoliating step, the treatment instrument 50 capable of scraping the inner wall 101 of the pancreatic cyst 100 is inserted into the decreased lumen 101, and the inner wall 101 is scraped by the treatment instrument 50. Therefore, since the inner wall 103 is scraped by the treatment instrument 50 while the lumen 101 of the pancreatic cyst 100 decreases in size, it is possible to scrape the inner wall 103 more efficiently.

In addition, compared to when the inner wall 103 of the decreased lumen 101 is scraped by the treatment instrument 50, it is difficult to collect tissue. However, the inner wall 103 of the lumen 101 may be scraped by the treatment instrument 50 after injecting the liquid 43 into the pancreatic cyst 100.

The above treatment instrument 50 has the brush-shaped treatment unit 51, and alternatively may have a sponge-shaped treatment unit. As long as the treatment instrument 50 has a configuration that can scrape the inner wall 101 of the pancreatic cyst 100, it is not particularly limited.

In addition, in the above Step S12, the treatment instrument configured to scrape the inner wall 101 of the pancreatic cyst 100 may have the following configuration.

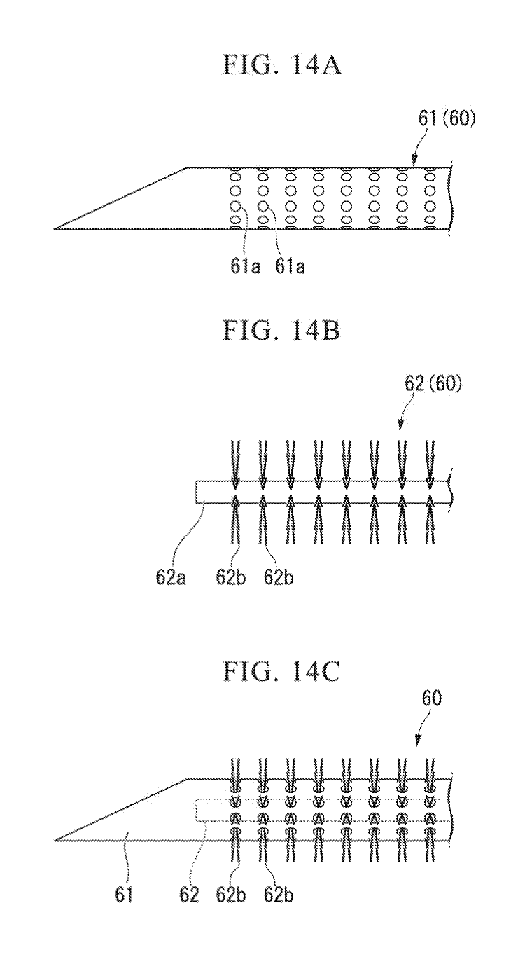

FIG. 14A to FIG. 14C show a treatment instrument 60, which is an example of the treatment instrument used in the method for collecting tissue according to the present embodiment. The treatment instrument 60 includes a needle tube 61 and a brush 62.

A plurality of side holes 61a are formed on an outer circumferential surface in the vicinity of a distal end of the needle tube 61. The brush 62 includes a wire-like member 62a and a plurality of brush bristles 62b that are disposed to correspond to the side holes 61a on an outer circumferential surface of the wire-like member 62a. By inserting the brush 62 into a lumen of the needle tube 61, the brush bristles 62b are projected from the side holes 61a of the needle tube 61. Accordingly, it is possible to increase the rigidity of the brush bristles 62b, and increase the force with which the treatment instrument 60 scrapes the inner wall 101 of the pancreatic cyst 100.

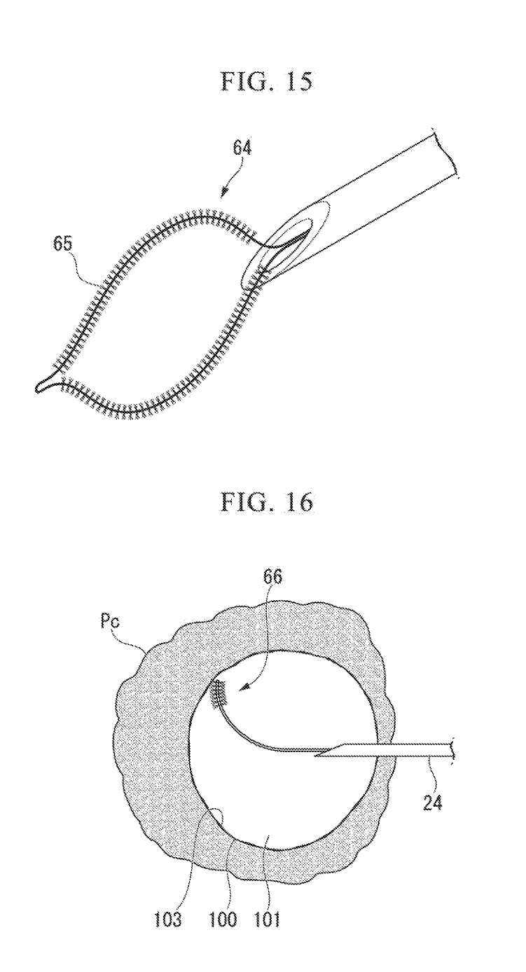

FIG. 15 shows a treatment instrument 64, which is another example of the treatment instrument used in the method for collecting tissue according to the present embodiment. The treatment instrument 64 includes a brush-shaped treatment unit 65 that is formed in a loop shape. Since the treatment unit 65 is formed to extend in a direction perpendicular to a projection direction, the treatment unit 65 can easily come in contact with the inner wall 101 of the pancreatic cyst 100.

FIG. 16 shows a treatment instrument 66, which is still another example of the treatment instrument used in the method for collecting tissue according to the present embodiment. The treatment instrument 66 is different from the treatment instrument 50 in that a distal end portion of the treatment instrument 66 has a tendency to bend. Therefore, the treatment instrument 66 can scrape a portion that is hard to scrape with the treatment instrument 50.

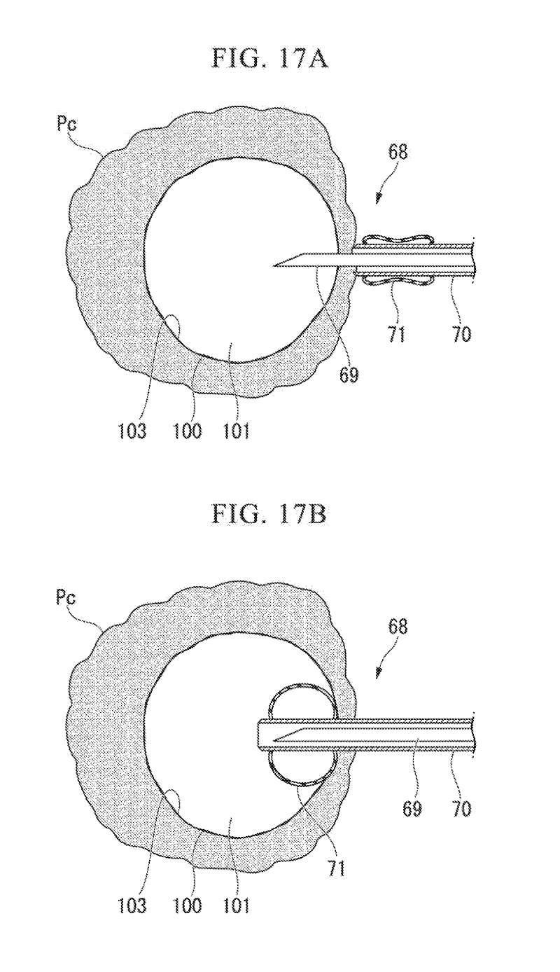

FIG. 17A and FIG. 17B show a treatment instrument 68, which is yet another example of the treatment instrument used in the method for collecting tissue according to the present embodiment. The treatment instrument 68 includes a needle tube 69, a sheath 70, and a balloon 71.

The needle tube 69 is inserted into a lumen of the sheath 70 such that the needle tube 69 can be advanced and retracted in the lumen of the sheath 70. A distal end of the sheath 70 is formed in a sharp shape, and can puncture the pancreatic cyst 100. The balloon 71 is inflatable and attached to an outer circumferential surface of a distal end portion of the sheath 70.

When the inner wall 101 of the pancreatic cyst 100 is scraped by the treatment instrument 68, the needle tube 69 first punctures the pancreatic cyst 100, and then the sheath 70 punctures the pancreatic cyst 100 such that the balloon 71 is positioned inside the pancreatic cyst 100. As shown in FIG. 17B, the balloon 71 is inflated. In this state, when the sheath 70 of the treatment instrument 68 is moved forward and backward and rotated, the balloon 71 scrapes the inner wall 101 of the pancreatic cyst 100.

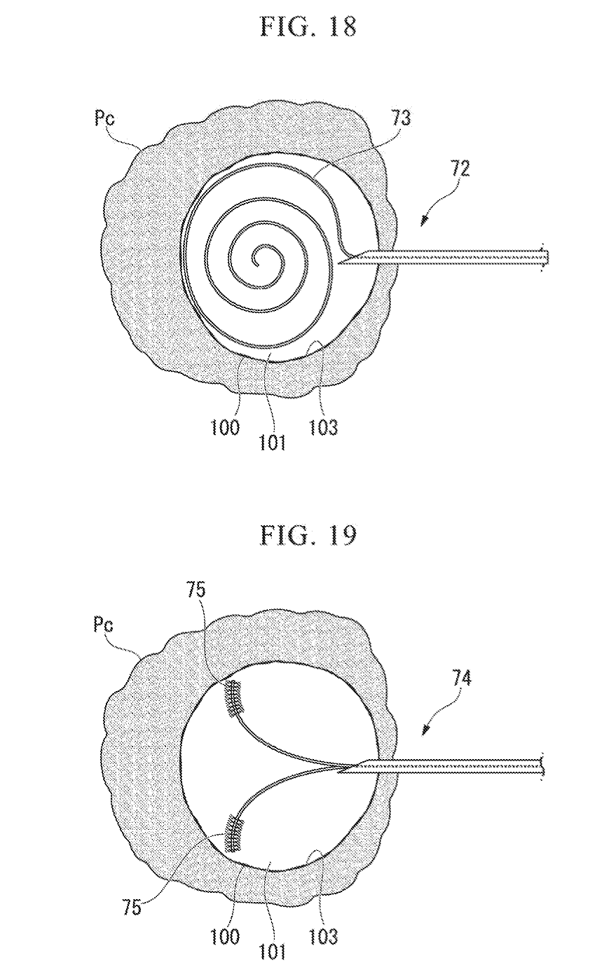

FIG. 18 shows a treatment instrument 72, which is yet another example of the treatment instrument used in the method for collecting tissue according to the present embodiment. The treatment instrument 72 includes a treatment unit 73 whose surface is configured to adsorb tissue of the pancreatic cyst 100. As shown in FIG. 18, when the treatment unit 73 is pushed into the pancreatic cyst 100, the treatment unit 73 comes in contact with the inner wall 101 of the pancreatic cyst 100, and adsorbs tissue of the inner wall 101.

FIG. 19 shows a treatment instrument 74, which is yet another example of the treatment instrument used in the method for collecting tissue according to the present embodiment. The treatment instrument 74 includes a pair of bifurcated treatment units 75 and 75 that have a brush shape. The pair of the treatment units 75 and 75 have a tendency to bend away from each other. By the pair of the treatment units 75 and 75, it is possible to scrape the inner wall 101 of the pancreatic cyst 100 more efficiently.

While exemplary embodiments of the present invention have been described above, the present invention is not limited to these embodiments. Additions, omissions, substitutions, and other modifications of the configuration can be made without departing from the scope of the present invention. The present invention is not limited to the above descriptions, and is only limited by the scope of the appended claims.

What is claimed is:

1. A method for collecting tissue comprising:
a body fluid removal step in which a body fluid inside a pancreatic cyst is moved to an outside of the pancreatic cyst;
an injecting step in which a liquid different from the body fluid is injected into the pancreatic cyst;
a collecting step in which the liquid is collected from the pancreatic cyst; and
a separating step in which tissue of an inner wall of the pancreatic cyst, which is contained in the liquid collected from the pancreatic cyst, is separated from the liquid.

2. The method for collecting tissue according to claim 1, further comprising
an exfoliating step in which the inner wall of the pancreatic cyst is scraped and the tissue is exfoliated from the inner wall,
wherein the exfoliating step is performed after the body fluid removal step, and the injecting step is performed after the exfoliating step.

3. The method for collecting tissue according to claim 2,
wherein, in the body fluid removal step, the body fluid is aspirated from the pancreatic cyst, and a lumen of the pancreatic cyst decreases in size, and
wherein, in the exfoliating step, a treatment instrument capable of scraping the inner wall of the pancreatic cyst is inserted into the decreased lumen, and the inner wall is scraped by the treatment instrument.

4. The method for collecting tissue according to claim 1,
wherein the liquid has a property of exfoliating the tissue from the inner wall of the pancreatic cyst.

5. The method for collecting tissue according to claim 4,
wherein the liquid is one of normal saline, a protease solution, and a surfactant solution.

6. The method for collecting tissue according to claim 1, further comprising
a perfusing step in which an inside of the pancreatic cyst is perfused with the liquid,
wherein the perfusing step is performed after the injecting step, and the collecting step is performed after the perfusing step.
